SEQUENCE LISTING (37 sequences)

SEQ ID NO: 1 - RNA, 10 nt, Homo sapiens
guguagagga

SEQ ID NO: 2 - RNA, 10 nt, Homo sapiens
cacgucucuu

SEQ ID NO: 3 - RNA, 12 nt, Homo sapiens
gauggagcaaga

SEQ ID NO: 4 - RNA, 12 nt, Homo sapiens
cuaccucguucu

SEQ ID NO: 5 - RNA, 32 nt, Homo sapiens
gugccguccgcccguccgucugcccgcaggca

SEQ ID NO: 6 - RNA, 23 nt, Homo sapiens
ggguggcacuugggggggcaggc

SEQ ID NO: 7 - RNA, 23 nt, Homo sapiens
ugucugcccgcaugccugccucu

SEQ ID NO: 8 - RNA, 32 nt, Homo sapiens
ugcagagggauccuguggcuggaagcgaggag

SEQ ID NO: 9 - DNA, 35 bp, Homo sapiens
gcaggcucccctcctcggcttccttcctctctgca

SEQ ID NO: 10 - RNA, 21 nt, Homo sapiens
aagccugcccggcuccucggg

SEQ ID NO: 11 - RNA, 24 nt, Homo sapiens
cacagguuuagacccagugugccu

SEQ ID NO: 12 - RNA, 27 nt, Homo sapiens
aggcaugcaggggagacgacucaggga

SEQ ID NO: 13 - RNA, 24 nt, Homo sapiens
ucccugagacccuuuaaccuguga

SEQ ID NO: 14 - RNA, 44 nt, Homo sapiens
ggagccggcugcgcgaggcgcccgccgaaggcccugcuguaaaa

SEQ ID NO: 15 - RNA, 44 nt, Homo sapiens
aucggccagugggccuucgggagggcggggggaagaugcugccu

SEQ ID NO: 16 - RNA, 31 nt, Homo sapiens
cgaggcgcccgccgaaggcccacuggccgaa

SEQ ID NO: 17 - RNA, 31 nt, Homo sapiens
aucggccagugggccuucgggagggcggggg

SEQ ID NO: 18 - RNA, 31 nt, Homo sapiens
cgaggcgcccggggugacgggugcuguaaaa

SEQ ID NO: 19 - RNA, 31 nt, Homo sapiens; identical to SEQ ID NO: 17

SEQ ID NO: 20 - RNA, 22 nt, Homo sapiens
uggcagugucuuagcugguugu

SEQ ID NO: 21 - RNA, 19 nt, Homo sapiens
caaagccaggcaggccaug

SEQ ID NO: 22 - RNA, 25 nt, Homo sapiens
uugcacugcccccugcuuccccagg

SEQ ID NO: 23 - RNA, 10 nt, Homo sapiens
uggcaguguc

SEQ ID NO: 24 - RNA, 19 nt, Homo sapiens
caaagggaccaccgccaug

SEQ ID NO: 25 - RNA, 25 nt, Homo sapiens
uugcacugccccacgguugaacagg

SEQ ID NO: 26 - RNA, 26 nt, Homo sapiens
uuggucacgccccacgguugaacagg

SEQ ID NO: 27 - RNA, 25 nt, Homo sapiens
uuggucacgccccugcuuggccagg

SEQ ID NO: 28 - RNA, 24 nt, Homo sapiens
gaacuggaagaagaaaaauuccca

SEQ ID NO: 29 - RNA, 24 nt, Homo sapiens
guuuuguuuuuuuuuuuccuaugc

SEQ ID NO: 30 - RNA, 24 nt, Homo sapiens
gaacuggaagaagaaaaauuccca

SEQ ID NO: 31 - RNA, 22 nt, Homo sapiens
guuuuguuuuuuuuuucuaugc

SEQ ID NO: 32 - RNA, 23 nt, Homo sapiens
gaacuggaagaagaaaauuccca

SEQ ID NO: 33 - RNA, 24 nt, Homo sapiens
guuuuguuuuuuuuuuuccuaugc

SEQ ID NO: 34 - RNA, 24 nt, Homo sapiens
gaacuggaagaagaaaaauuccca

SEQ ID NO: 35 - RNA, 23 nt, Homo sapiens
guuuuguuuuuuuuuuccuaugc

SEQ ID NO: 36 - RNA, 24 nt, Homo sapiens
gaacuggaagaagaaaaauuccca

SEQ ID NO: 37 - RNA, 24 nt, Homo sapiens
guuuuguuuuuuuuuuuuccuaug

(19) United States
(12) Patent Application Publication
(10) Pub. No.: US 2019/0325987 A1
(43) Pub. Date: Oct. 24, 2019

(54) Direct Interaction Between 5' UTR and 3' UTR Enhances miRNA Translation Repression

(71) Applicant: INHAN LEE, ANN ARBOR, MI (US)

(72) Inventor: INHAN LEE, ANN ARBOR, MI (US)

(21) Appl. No.: 16/390,658

(22) Filed: Apr. 22, 2019

Related U.S. Application Data
(60) Provisional application No. 62/660,570, filed on Apr. 20, 2018.

(51) Int. Cl. G16B 20/30; G16B 15/10; G16B 15/30; C12Q 1/6858; C12Q 1/6827

(52) U.S. Cl. CPC G16B 20/30; G16B 15/10; G16B 15/30; C12Q 2600/166; C12Q 1/6827; C12Q 2600/156; C12Q 1/6858

(57) ABSTRACT

A computer implemented method for evaluating if a mRNA nucleotide sequence can form a triBridge complex. The miRNA-mRNA triBridge complex is a complex formed from the mRNA nucleotide sequence and the miRNA nucleotide sequence that includes the UTR-UTR zipper and a miRNA bridge. A biological sample can be obtained from a subject to determine the presence of single nucleotide polymorphisms in the mRNA nucleotide sequence that forms UTR-UTR zipper and a miRNA bridge.

### CROSS-REFERENCE TO RELATED APPLICATIONS

This application claims the benefit of U.S. provisional Application No. 62/660,570 filed Apr. 20, 2018, the disclosure of which is incorporated in its entirety by reference herein.

### SEQUENCE LISTING

The text file MIRC0106_ST25 of size 5 KB created Apr. 22, 2019, filed herewith, is hereby incorporated by reference.

### TECHNICAL FIELD

In at least one aspect, the present invention relates to methods of predicting miRNA targets and integrative biomarkers from miRNA and mRNA expression patterns. Such methods find use in research, diagnostic and therapeutic settings (e.g., to discover targets, drugs, diagnostic products, etc.).

### BACKGROUND

Identifying disease-relevant pathways using large genome-wide datasets pose distinct challenges. The data is vast, diverse, and inherently complex, being derived from DNA, mRNA, non-coding RNA, and protein levels, so that little progress has been made towards combining multiple platform datasets. Even dealing with one platform, the sheer bulk of data forces researchers to focus on previously known genes, rather than new genetic mechanisms, due to a lack of tools generating pathways in a hypothesis-free manner. It has become clear that most diseases are due to a combination of genetic and environmental factors and that genetic factors themselves are due to combined effects of multiple genes rather than one bad gene, especially for diseases with complex causes and symptoms. Seeing how a single miRNA could regulate several mRNAs and a single mRNA could be regulated by several miRNAs, we set out to identify multiple disease-relevant genes by combining expression data from two platforms, miRNA and mRNA.

Most miRNAs are transcribed similarly to other protein-coding genes, processed by Drosha enzyme into a hairpin-shaped precursor which is transported into the cytosol for further processing by Dicer enzyme until a single strand of mature miRNA is loaded into RNA-induced silencing complex (RISC), making a functional miRNA-protein complex (miRNP). Translation repression by a miRNA occurring in a sequence-specific way can present mild to significant mRNA degradation, probably due to the secondary effect of other enzymes after localization of mRNA-miRNP complexes in the cytosolic loci. As human mature miRNAs number 722 (including 167 star-named sequences) in miRBase version 10.0 as of 2008 and all human mRNAs total about 20,000, one miRNA may regulate many genes in a single biological context. In fact, many miRNA target-finding programs predict several hundreds to thousands of target genes for one miRNA. However, many of these predicted targets turn out to be false positives, constituting a major hurdle in understanding miRNA function.

Also relevant is the mRNA canonical translation initiation which includes binding of PABP (poly(A)-binding protein) to the 3′ poly(A) tail of mRNA and eIF4G (a subunit of the cap binding protein complex, eukaryotic initiation factor 4F complex), thus bringing the tail of mRNA to the 5′ start site of mRNA^{1,2}. Untranslated regions (UTRs) of mRNAs function in translation regulation such as 3′ UTRs through microRNAs (miRs) and 5′ UTRs through secondary structures or upstream AUG sequences.

Accordingly, there is a need for improved method of evaluating mRNA and miRNA expression patterns.

### BACKGROUND

In at least one aspect, a method for determining is a candidate mRNA nucleotide sequence can form a miRNA-mRNA triBridge complex being is provided. The method includes steps of:

a) executing by a computer a step of receiving into computer memory data identifying an mRNA nucleotide sequence representing a gene or portion thereof, the mRNA nucleotide sequence has an upstream UTR region that is upstream of translation start site, a downstream UTR region that is downstream of translation stop site, and an open reading frame;

b) executing by the computer a step of evaluating the upstream UTR region for a first nucleotide UTR subregion that can stably hybridize to a second nucleotide UTR subregion in the downstream UTR region such that if the first UTR nucleotide subregion can stably hybridizes the second UTR nucleotide subregion a first hybridization region is identified, the first hybridization region being a UTR-UTR zipper;

c) executing by the computer a step of receiving into computer memory data identifying a miRNA nucleotide sequence, the miRNA nucleotide sequence having a 5′ miRNA section and a 3′ miRNA section;

d) executing by the computer a step of evaluating the upstream UTR region for a third UTR nucleotide subregion that is capable of stably hybridizing to at least of a subregion of the 3′ miRNA section or at least a portion of the 5′ miRNA section such that if the third UTR nucleotide subregion can stably hybridizing to at least of a subregion of the 3′ miRNA section or at least a subregion of the 5′ miRNA section a second hybridization region is identified;

e) executing by the computer a step of evaluating the downstream UTR region for a fourth UTR nucleotide subregion that is capable of stably hybridizing to at least of a subregion of the 5′ miRNA section or at least a subregion of the 3′ miRNA section such that if the fourth UTR nucleotide subregion can stably hybridizing to at least of a subregion of the 5′ miRNA section or at least a subregion of the 3′ miRNA section a third hybridization region is identified; and'

f) executing by the computer a step of identifying the mRNA nucleotide sequence as a candidate for forming a miRNA-mRNA triBridge complex, the miRNA-mRNA triBridge complex being a complex formed from the mRNA nucleotide sequence and the miRNA nucleotide sequence that includes the first hybridization region, the second hybridization region and the third hybridization region where the second hybridization region and the third hybridization region form a miRNA bridge.

### BRIEF DESCRIPTION OF THE DRAWINGS

FIGS. 1A and 1B provide a computer implemented method for determining if a candidate mRNA nucleotide sequence can form a miRNA-mRNA triBridge.

FIG. 2 is a schematic of a computer system that can implement the method of FIG. 1.

FIGS. 3A, 3B, 3C, and 3D: (A) miBridge and (B) triBridge models and abundantly existing 5U=3U interaction sites. Based on the 5U interaction separation defined in C, the dotted line in D shows the real 5U=3U interaction frequencies, whereas the solid line shows those from the shuffled 3U sequence.

FIG. 4. Table 1 shows a summary of the numbers of input and output from the miBridge analysis as well as the UTR to UTR analysis. Row one lists the totals for information pertaining to the miBridges, while rows two and three list the totals for information pertaining to the 3′UTR downstream vs. the 5′ UTR and 3′ UTR upstream vs. the 5′ UTR, respectively. The first column lists of the total number of transcripts that we tested, while the second column lists the number of transcripts that were found to have at least one miBridge site or UTR to UTR site. Column three lists the total number of miRNA that we tested, while column four lists the number of miRNA that were found to make at least one miBridge site or have at least one UTR to UTR site down/upstream of their miBridge site. The fourth column gives the number of miRNA to transcript valid combinations found during the miBridge analysis. The last column lists the total number of UTR to UTR interaction sites discovered.

FIGS. 5A, 5B, 5C, and 5D: 5U=3U interaction examples from the experimentally validated miR-target pairs. (A) Table 2 UTR to UTR interaction output examples that have been experimentally verified transcript+miRNA relations through reporter assay or western blot. SEQ ID NO: 1, SEQ ID NO: 2, SEQ ID NO: 3, SEQ ID NO: 4 are listed in the table. (B) Illustration of miBridge interaction between NM_016938-EFEMP2 and miR-346. (C) Illustration of miBridge interaction between NM_005567-LGALS32BP and miR-596. (D) Illustration of miBridge interaction between NM_006465-ARID3B and miR-125a-5p.

FIGS. 6A, 6B, and 6C: 5U=3U interaction example from the experimentally validated miR-target pairs. (A) shows the schematics of AXIN2 and miR-34a-5p triBridge sites; (B) gain of miR-34a function with the increase of the number of UTR zipper interaction site; and (C) loss of miR-34a function with removing the UTR zipper interaction site, without changing miR-34a-5p and target gene interaction sequences.

FIGS. 7A, 7B, 7C, 7D, and 7E: 5U=3U interaction example from the experimentally validated miR-target pairs. (A) a schematic of UTR zipper mutant and miR bonding site. Figures (B) luciferase activity (Luciferase/renilla) abundance using wt and zipper mutant sequences in UTRs; (C) luciferase activity using wt and MBS mutant sequences in UTRs; (D) relative transcript abundance (Luciferase/internal control) using wt and zipper mutant sequences in UTRs; and (E) luciferase activity and relative transcript abundance (Luciferase/internal control) using wt and MBS mutant sequences in UTRs.

FIG. 8: SNPs in 5U=3U interaction sites of AXIN2 in colon cancer patients. Hybridization characteristics and patient information for each SNPs were shown together.

### DETAILED DESCRIPTION

The invention, both as to its exemplary organization and illustrative manner of operation, together with further objects and advantages thereof, may best be understood with reference to the following description, taken in connection with the accompanying drawings, in which:

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “miRNA-mRNA triBridge complex” refers to a complex between a mRNA nucleotide sequence and a miRNA that includes a UTR-UTR zipper and a miRNA bridge. Sometimes herein “miRNA-mRNA triBridge complex” is referred to as “triBridge complex” or as “triBridge” or as “triBridge interaction.”

The term “miRNA bridge” refers to a complex between mRNA nucleotide sequence and a miRNA in which the miRNA bridges the upstream URT of the mRNA nucleotide sequence and the downstream UTR of the mRNA nucleotide sequence. Aspects of the miRNA bridge are set forth in U.S. Pat. No. 8,768,630; the entire disclosure of which is hereby incorporated by reference.

### Abbreviations

“MBS” means miRNA binding site.

“miR” means miRNA.

“miRNP” means miRNA-protein complex.

“PABP” means (poly(A)-binding protein).

“UTR” means untranslated region.

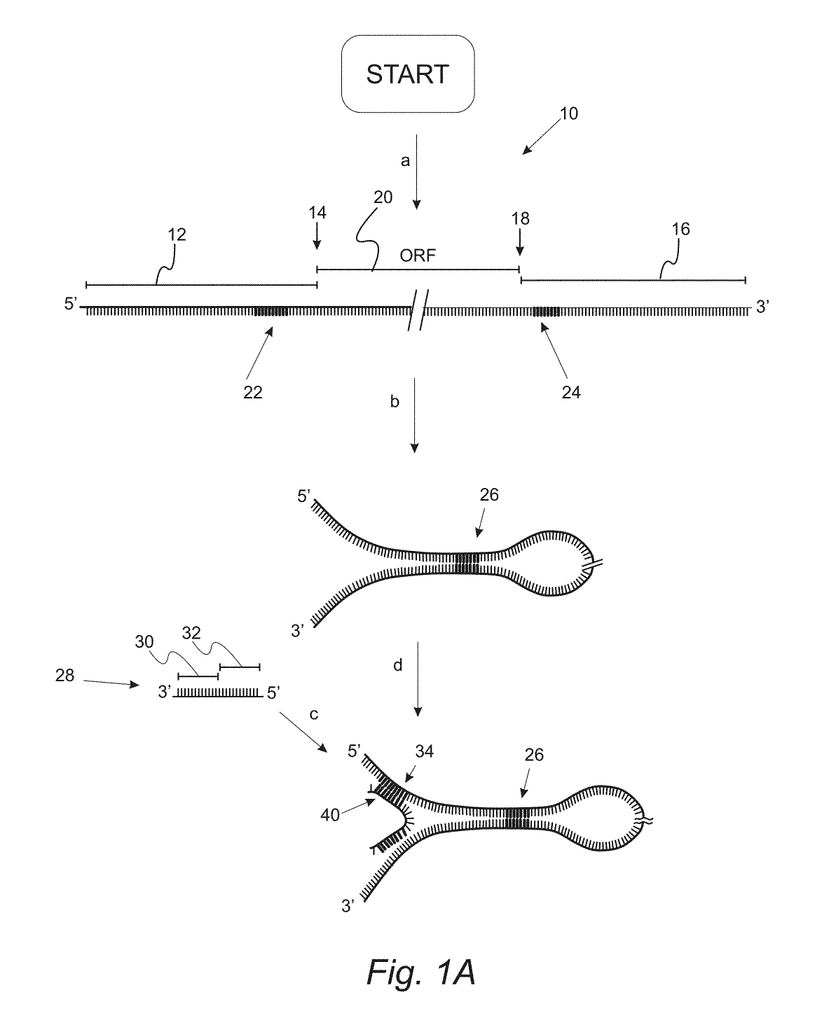

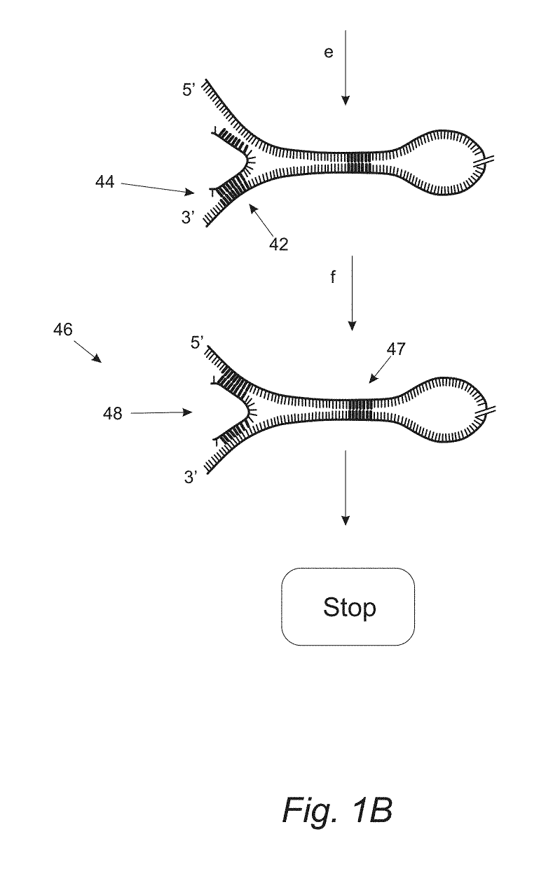

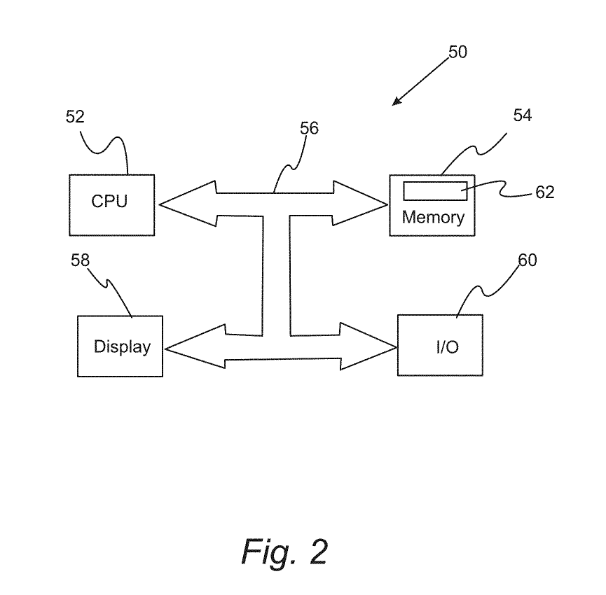

With reference to FIG. 1, a computer implemented method is schematically illustrated. Typically, the steps of the method are executed by a computer as illustrated in FIG. 2. The includes step a) of receiving (e.g., into computer memory) data identifying an mRNA nucleotide sequence 10 representing a gene or subregions thereof. Candidate mRNA nucleotide sequences can be downloaded from www.ncbi.nlm.nih.gov/. Characteristically, the nucleotide sequence has an upstream UTR region 12 that is upstream of translation start site 14, a downstream UTR region 16 that is downstream of translation stop site 18, and an open reading frame 20. Upstream UTR region 12 is evaluated (e.g., by the computer) in step b) for a first UTR nucleotide subregion 22 that stably hybridizes to a second UTR nucleotide subregion 24 in the downstream UTR region 16. If the first UTR nucleotide subregion 22 can stably hybridize to the second UTR nucleotide subregion 24, first hybridization region 26 can be identified. Typically, first hybridization region 26 is referred to as a UTR-UTR zipper. Typically, the first hybridization region the first UTR nucleotide subregion in in an opposite direction to the second UTR nucleotide subregion with respect to 5′ to 3′ direction.

In step c), data identifying at least one microRNA (miRNA) nucleotide sequences is received (e.g., into computer memory). Characteristically, the at least one miRNA sequence 28 includes a 5′ miRNA section 32 and a 3′ miRNA section 30. Candidate miRNA sequences for mRNA nucleotide sequence 10 can be downloaded from mirbase.org/ftp.shtml or can be design by inspecting the upstream and downstream regions as set forth below.

In step d), the upstream region 12 is evaluated (e.g., by the computer) for a third nucleotide subregion 34 that is capable of stably hybridizing to at least of a subregion of the 3′ miRNA section 30 or the 5′ miRNA section 32 if the third nucleotide subregion can stably hybridizing to at least of a subregion of the 3′ miRNA section or the 5′ miRNA section a second hybridization region 40 is identified. In step e), the downstream region 16 is evaluated for a fourth nucleotide subregion 42 that is capable of stably hybridizing to at least of a subregion of the 5′ miRNA section 32 or the 3′ miRNA section 30. If the fourth nucleotide subregion 42 can stably hybridizing to at least of a subregion of the 5′ miRNA section 32 or the 3′ miRNA section 30 a third hybridization region 44 is identified.

In step f) nucleotide sequence is identified as a candidate for forming a miRNA-mRNA triBridge complex 46 which is a combination the first hybridization region 26, the second hybridization region 40 and the third hybridization region 44. Characteristically, miRNA sequence 28 in the microRNA-mRNA triBridge complex bridges the upstream region to the downstream region and is referred to as a miRNA bridge. Moreover, in the microRNA-mRNA triBridge complex when 3′ miRNA section 30 stably hybridizes to upstream region 12, the 5′ miRNA section 32 stably hybridizes to downstream region 16. Alternatively, in the microRNA-mRNA triBridge complex when 5′ miRNA section 32 stably hybridizes to upstream region 12, the 3′ miRNA section 30 stably hybridizes to downstream region 16. Therefore, a miRNA-mRNA triBridge complex 46 includes a UTR-UTR zipper 47 and a miRNA bridge 48.

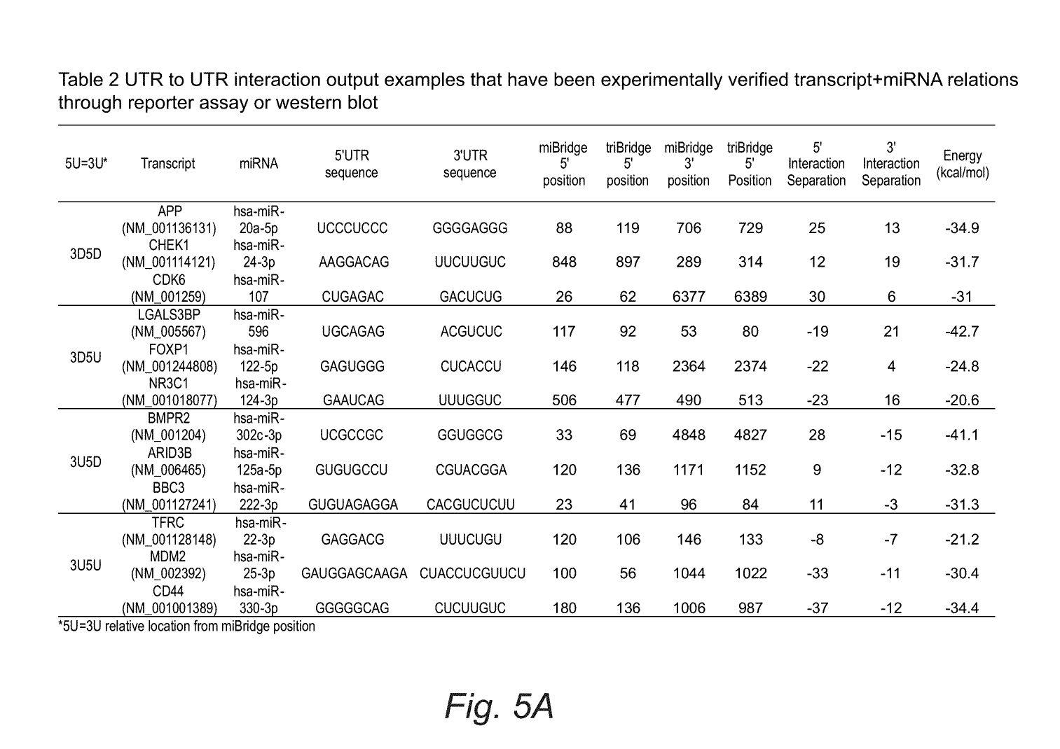

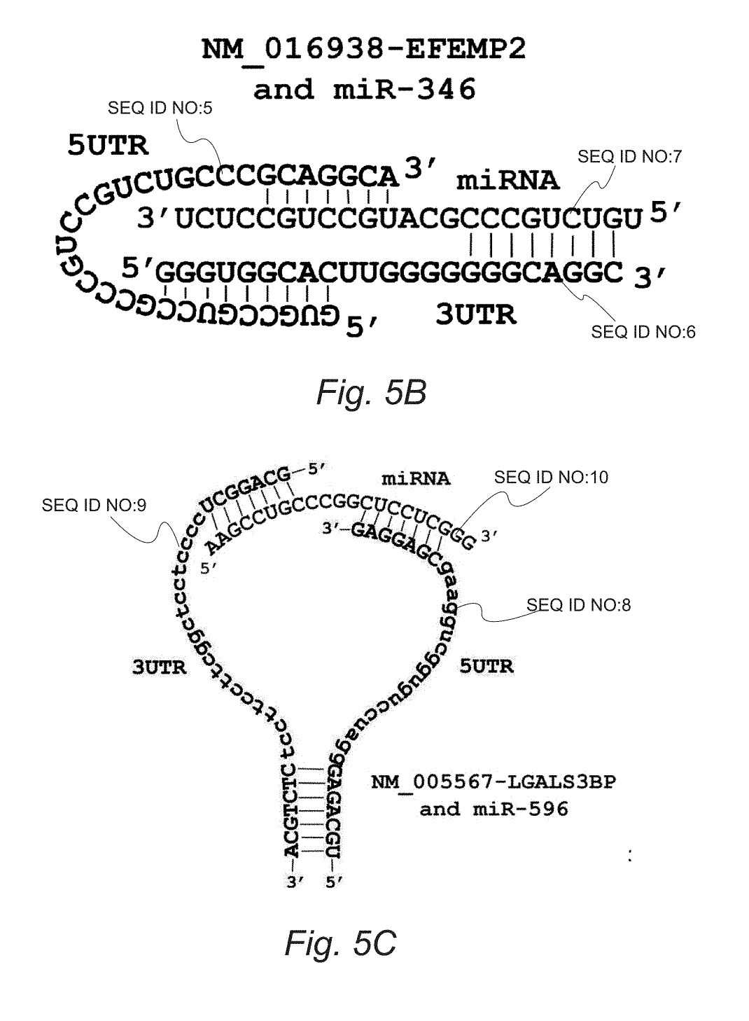

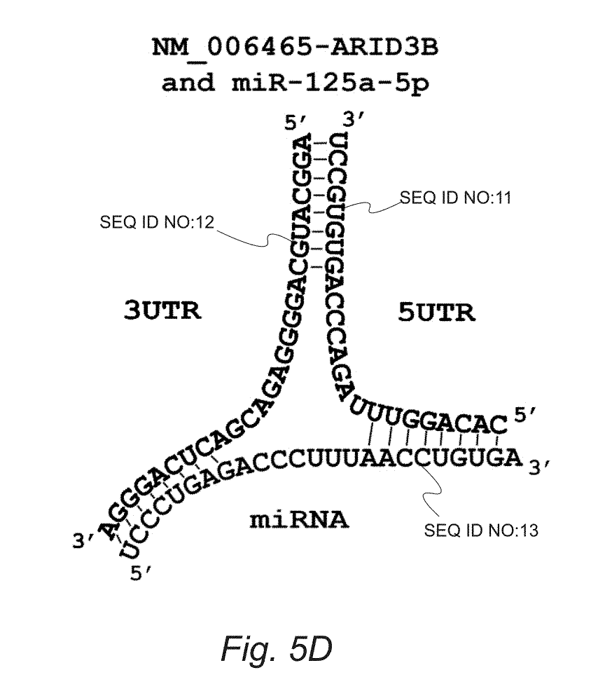

It should also be appreciated that different configurations of a miRNA-mRNA triBridge complex 46 are possible depending if a miRNA interaction (i.e., the miRNA bridge) is upstream of the first hybridization region or downstream of the first hybridization region; or upstream of the second hybridization region or downstream of the second region. (see, FIGS. 5B, 5C, and 5D).

As set forth above, miRNA sequence 28 includes a 3′ miRNA section 30 and a 5′ miRNA section 32. Candidate miRNA sequences can be downloaded from http://mirbase.org/ftp.shtml. Alternatively, miRNA sequences be designing a miRNA that can simultaneously hybridize to upstream region 12 and downstream region 16. In a refinement, such a miRNA can be formulated by inspection by constructing a nucleotide sequence with a 3′ miRNA section or subregion thereof perfectly complementary to a subregion of upstream region 12 and 5′ miRNA section or subregion thereof perfectly complementary to a subregion of downstream region 16.

As set forth above, a first hybridization region 26, second hybridization region 40, and the third hybridization region 44 each call for stable hybridization. Typically, these hybridization regions include in increasing order of preference at least 5, 6, 7, 8, 9, or 10 nucleotide base pairs. In a further refinement, the hybridization regions include in increasing order of preference at most 50, 30, 20, 18, 15 or 10 nucleotide base pairs. For example, the hybridization regions can include 5 to 15 nucleotide base pairs. Typically, stable hybridization is determined by a degree of complementariness with perfectly complementary subregions are most stable. In a refinement, complementary subregions include at most 2 mismatches. In another refinement, stable hybridization is determined by thermodynamic criteria. Specifically, the change ΔG in Gibbs free energy for the interaction of a subregion of 5′ miRNA section 32 with sub-regions of section 34 is evaluated with interactions having ΔG less than a predetermined value being identified as candidate sites for in vivo interactions. In a further refinement, ΔG for these hybridizations is less than about −10 kcal/mol. In still a further refinement, ΔG for these hybridizations is less than about −13 kcal/mol. The thermodynamic calculation may be carried out using the RNAhybrid™ software available from bibiserv.techfak.uni-bielefeld.de/rnahybrid/.

In a variation, the method set forth above is repeated for a plurality of mRNA nucleotide sequences 10 and/or a plurality of miRNA sequences 28 to identify the ability of the mRNA nucleotide sequences to form triBridge complexes. In a refinement, the nucleotide sequence 10 is a gene associated with a cancer or risk of developing a cancer. Examples of such cancers include, but are not limited to, lung cancer, breast cancer, and colon cancer (e.g., colon adenocarcinoma). Examples of such genes include, but are not limited to the WNT1 gene and the AXIN2 gene.

With reference to FIG. 2, a computer system for implementing the method set forth above, and in particular steps a)-f) of FIG. 1 is provided. System 50 of the present invention includes central processing unit (CPU) 52, memory 54, and input/output interface 56. Computer system 50 communicates with display 58 and input devices 60 such as a keyboard and mouse via interface 56. In one variation, memory 54 includes one or more of the following: random access memory (RAM), read only memory (ROM), CDROM, DVD, flash drive, disk drive, tape drive and the like. The method of various embodiments is implemented by routine 62 that is stored in memory 54 and executed by the CPU 52. In another variation, a non-transitory computer readable medium embodying a program of instructions executable by a processor to perform the method steps set forth above is provided. Specifically, the computer readable medium is encoded with instructions for the steps of the methods of the invention. Example of useful computer readable media include, but are not limited to, hard drives, floppy drives, CDROM, DVD, optical drives, random access medium, and the like.

In another variation, a biological sample is obtained from a subject. Examples of biological samples include, but are not limited to, blood, plasma, cell free plasma, serum, CSF, urine, feces, saliva, biopsy samples, tissue, skin, hair, tumor, PAP smears, moles, warts, and the like. In a refinement, a patient's biological sample is evaluated for single nucleotide polymorphisms in the zipper region or in the miRNA binding regions. The first hybridization region, the hybridization region, and the third hybridization region are each independently evaluated for single nucleotide polymorphisms. Specifically, the mRNA sequence or corresponding DNA sequence is evaluated for polymorphisms in the first nucleotide UTR subregion and/or the second nucleotide UTR subregion and/or the third nucleotide UTR subregion and/or the fourth nucleotide UTR subregion.

In another embodiment, a candidate mRNA sequence is evaluated for the presence of first UTR nucleotide subregion 22 and second UTR nucleotide subregion 24 that can form UTR-UTR zippers a set forth above via stable hybridization. First UTR nucleotide subregion 22 that stably hybridizes to a second UTR nucleotide subregion 24 can then be evaluated for SNPs. For candidate sequences that are associated with cancer (e.g., to the WNT1 gene and the AXIN2 gene) SNP in these subregions can be predictive of prognosis. In a variation, such mRNAs having UTR-UTR zippers are evaluated to regions that can form miRNA bridges as set forth above.

It should be appreciated that although the methods set forth above as cast in steps involving RNA, DNA sequences can also be used since the transformation of DNA sequences to mRNA sequences is a well-know transformation.

The following examples illustrate the various embodiments of the present invention. Those skilled in the art will recognize many variations that are within the spirit of the present invention and scope of the claims.

Here we show that direct interactions between the 5′ UTR and 3′ UTR exist preferentially near miR interaction sites for both 5′ UTR and 3′ UTR and that these UTR-UTR interaction zippers enhance miR function in a sequence-specific fashion in reporter experiments for WNT1 and AXIN2 genes. We found that SNPs³ for both WNT1 and AXIN2 exist in these UTR-UTR zippers and are highly specific to ethnicity. Furthermore, three cancer types from TCGA datasets presented more SNPs in these UTR zipper sites than in other UTR regions of WNT1 and AXIN2. Interestingly, colon adenocarcinoma (COAD) has a higher incidence of AXIN2 UTR-UTR zipper SNPs than breast or lung cancers, which aligns well with colorectal cancer currently being the only cancer with AXIN2 gene-phenotype relationships^{4,5}, based on OMIM (Online Mendelian Inheritance in Man). We also found a higher death rate for COAD patients with SNPs in AXIN2 UTR-UTR zipper sites, possibly due to reduced miR-34a-5p regulation of AXIN2. Our finding demonstrates a new translation mechanism involving direct 5′ UTR and 3′ UTR interactions, which seem to exist universally, at least in computational analysis. These direct UTR-UTR interactions may facilitate translation repression of miRs which interact with 3′ UTR, thus increasing the likelihood of interaction between the 3′-end of miR and the 5′ UTR^{6,7}. Finally, our finding may aid in discovering disease-associated SNPs in UTR regions, which have until now been limited to miR-interacting 3′ UTR sites^{8,9}.

Accumulating deep-sequencing data is rapidly expanding RNA kingdom, yielding insights of RNA functions in regulation of gene expression and of RNA-RNA interaction networks. Among these, miRNAs (miRs) are well-annotated and studied non-coding RNAs functioning as translational repression and/or mRNA degradation. While miRs regulate target RNAs through direct base-pairing, miR functions can be modulated through competitive interactions (ceRNA)^{10,11} or interaction with naturally existing sponges (circRNA)^{12,13}.

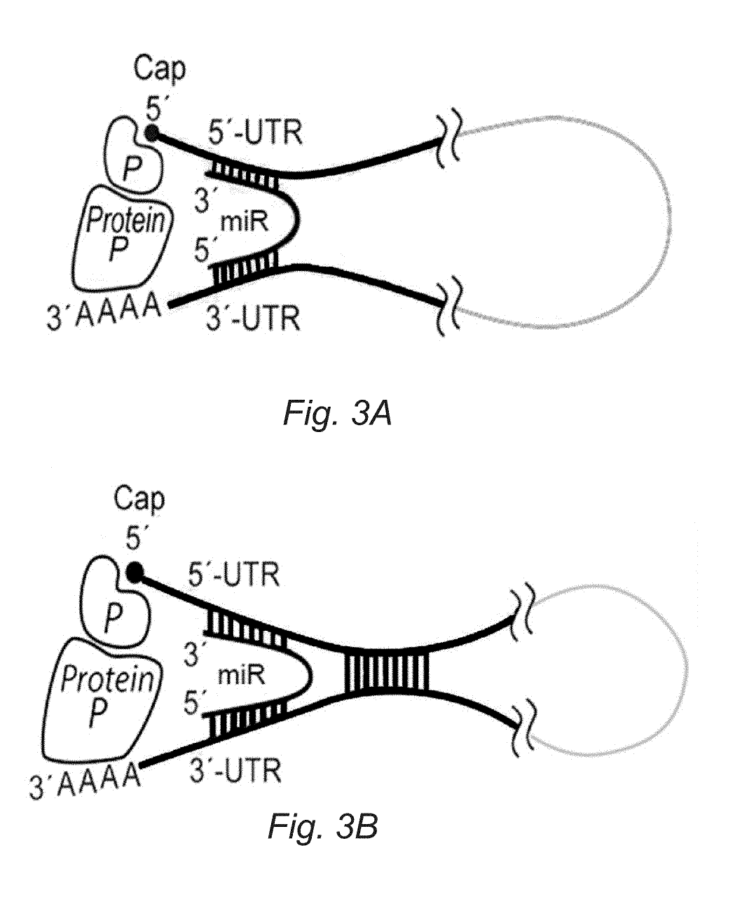

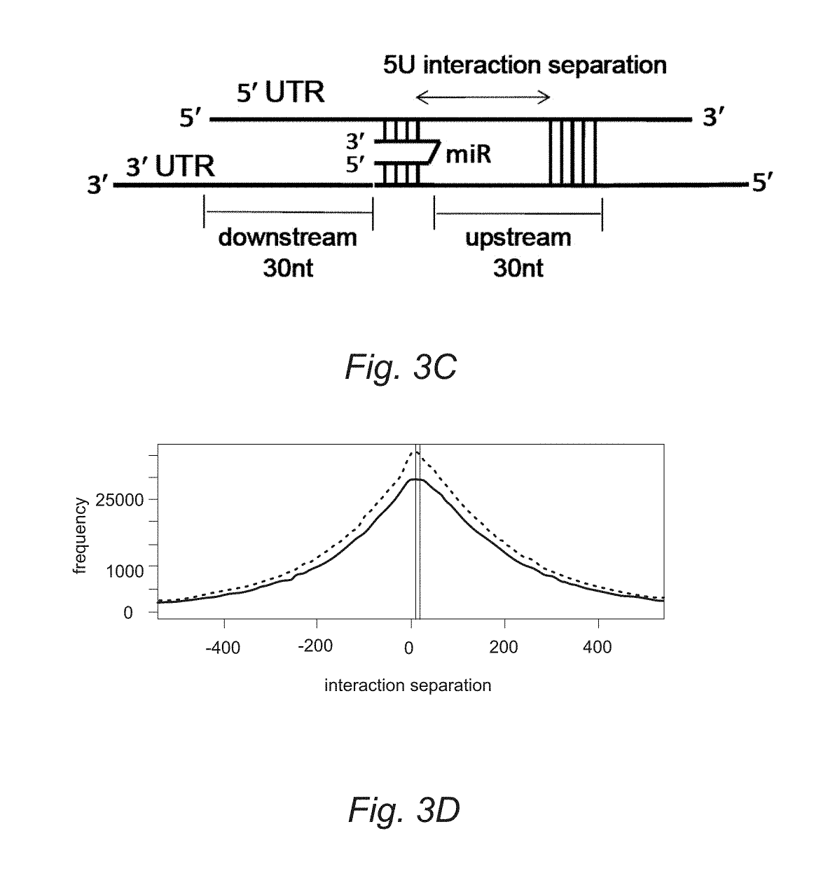

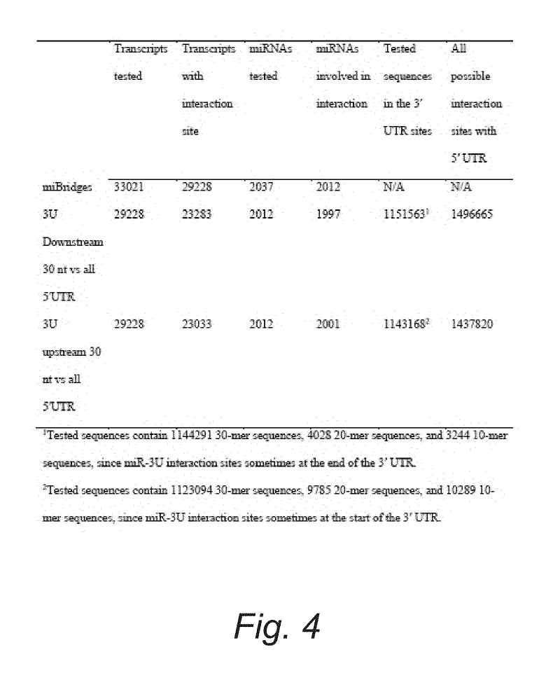

Though miRNA can interact with various regions of a target transcript, most miR-target interaction occurs through the 3′ UTR of a target and the 5′-end (seed region) of a miRNA^{14,15}. Unlike siRNA (small interfering RNA), wherein central regions of small RNA (9 to 12^{th} position from the 5′-end) can facilitate endonuclease function of the PIWI domain^{16,17}, many animal miRNAs lack central region interaction with the 3′ UTR^{14,18}. Considering that the usual translation process can bring the 5′ UTR and the 3′ UTR nearby through poly(A)-binding protein (PABP) interaction with the multiple cap binding proteins, we showed that the loosely bound 3′-end of a miRNA can directly interact with the 5′ UTR′, possibly forming a miRNA bridge (miBridge, FIG. 3A). Together with miRNA interacting proteins (miRNPs), this configuration is useful for blocking ribosome scanning when the endonuclease site is not occupied, leading to translation repression. We found that initially identified C. elegans miRNAs have such target sites (3′-end of miRNA interaction with the upstream of coding region) and that the miBridge interaction has a stronger interaction than miRNA interaction with 3′ UTR alone, confirming that such interactions are thermodynamically preferable. Also, since AGO crystal structures presented an additional positive groove near the middle of the main positive grooves accommodating double stranded RNAs^{19,20}, RNA cross-over is structurally available. We also found that the 3′-end of miRNAs have significant 5′ UTR interaction sites (especially between the 3′-end of conserved miRNA and the upstream AUG motifs in the 5′-UTR⁶) as compared with randomly shuffled miRNA sequences and that such sequence-specific interactions are functional⁷. Using miRBase release 19 and hg19 RefSeq transcript downloaded from UCSC genome browser (May 2013), we found 88.5% of the transcripts possess at least one potential miBridge site, meaning that both of the transcripts's UTRs possessed sites that could interact with the two ends of a single particular miRNA. Conversely, 98.7% of the miRNA which we tested has a potential triBridge interaction site with at least one transcript.

Based on miBridge configuration, we recognized that direct interaction between 5′ UTR and 3′ UTR strands might occur. This 5′ UTR-3′ UTR zipper (5U=3U) will form three-way bridge type interactions (triBridge), as shown in the FIG. 3B, further increasing the ability of miRNA to stall ribosomes. To check if 5U=3U interaction sites exist near the miBridge sites, we took 30 nt sequences upstream and downstream of the 3′ UTR miBridge site (FIG. 3C) and checked for possible interaction sites in the whole 5′ UTR sequences, as described in detail in Methods. 79.7% of the 30 nt sequences downstream of the 3′ UTR have at least one 5U=3U interaction site and 78.8% of the upstream sequences have at least one site. In total, 99.5% of miRNA with a miBridge interaction has a 5U=3U site 30 nt either upstream or downstream from the 3′-UTR miBridge site (Table 1). We define 5U interaction separation as the distance between the 5U=3U and 5U=miRNA interaction sites in the 5′ UTR (FIG. 3C). The FIG. 3D dashed line shows the occurrence distribution of the 5U=3U interaction sites as a function of 5U interaction separation. To test its significance, we randomized the 30 nt up or downstream 3′ UTR sequences by keeping their sequence contexts but changing the sequence orders and checked their interactions with the 5′ UTR (occurrence distribution, FIG. 3D solid line). FIG. 3D clearly shows that real 3′ UTR sequences have significantly more interaction sites with the 5′ UTR, especially near the miBridge interaction sites. The mean interaction separation of true 3′ UTR vs. entire 5′ UTR regions is 17 nt, significantly shorter than those of randomized sequences (t-test p-value <0.00001). As most differences are found at regions immediately adjacent the miBridge site and the 3U interaction separations were essentially between 1 and 24, we focused on 5U interaction separations between 10 and 20 nt. The true 3′ UTR downstream sequences have 38,489 occurrences of an interaction separation between 10 and 20 nt, significantly higher than for randomized sequences (t-test p-value <0.00001). Therefore, triBridge interaction can theoretically exist for about 70% of the transcripts and 97% of miRNAs we tested in a sequence-specific way.

To investigate if triBridge sites exist among the known miR and target pairs, we downloaded pairs experimentally verified through reporter assay or western blot from miRTarBase release 4.5 and searched for miBridge sites in the 5′ UTR and 3′ UTR and having 5U=3U interaction with 30 nt next to the 3U=miR sites. We found that 546 out of 3108 miRTarBase with strong evidence entries have triBridge interactions. Interestingly, we found that 458 of such triBridge miR-target pairs have less than 100 nt 5U interaction separation. Therefore, 17.6% of known miR-target pairs had triBridge type interactions with 3U interaction separation less than 24 nt; and 83.8% of them had 5U interaction separation less than 100 nt. FIG. 5A shows 12 examples of strong hybridization energy and 5U=3U near the miBridge sites among well-known miRNA or mRNA, grouped according to whether the 5U=3U sites are upstream or downstream of the miBridge sites. Depending on the 5U=3U site location, the strand orientation for interactions varies, as depicted in FIG. 5B-D. When 5U=3U sites are both down or up (FIG. 5B) in relation to the miBridge sites, the possible configuration is characterized by the wrapping of one UTR around the other in order to orient the UTRs properly to each other. This may be more easily achieved when one of the interaction separations is greater than the other. FIG. 5C shows when 3U downstream interacts with 5U upstream from the miBridge sites and vice versa in FIG. 5D, where the interaction separations of 5U and 3U may prefer similar distances. In all three configurations, miRNA can securely lock configuration, preventing a ribosome from reaching the start codon.

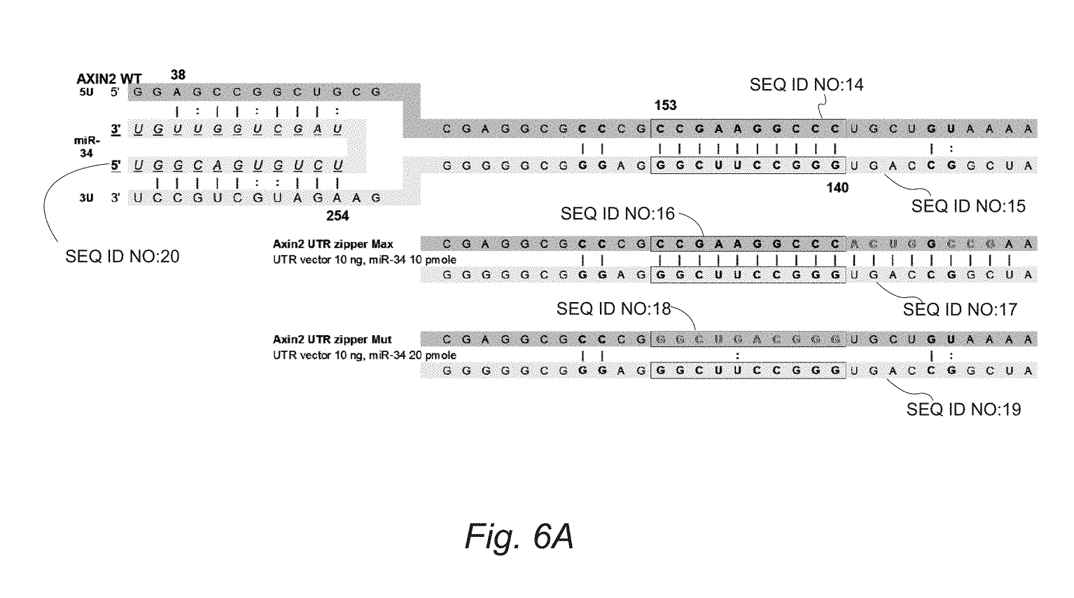

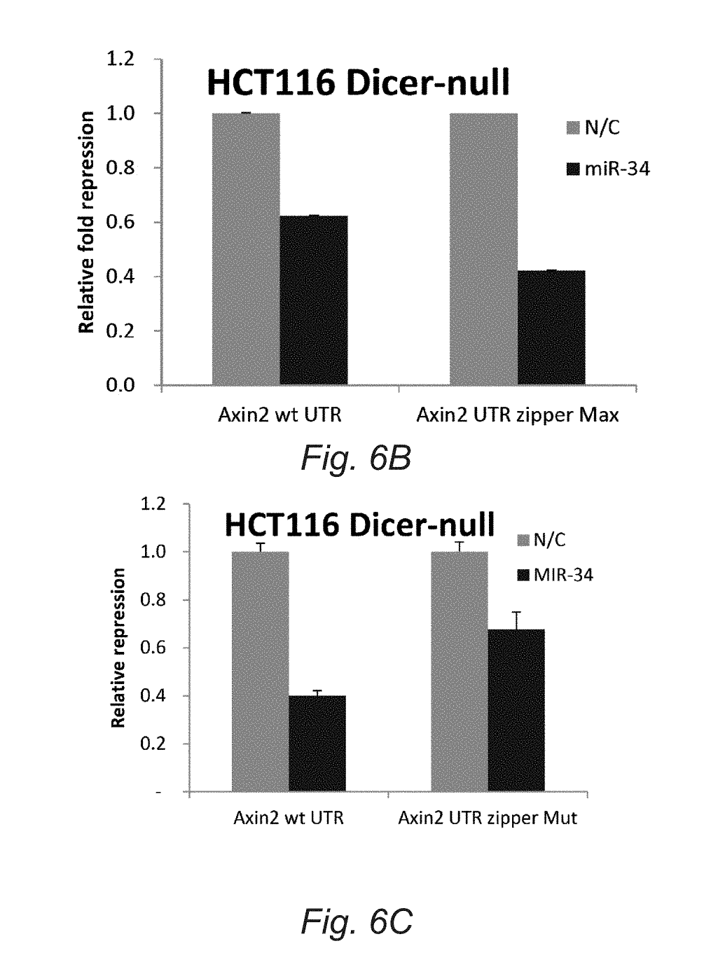

To validate 5U=3U interaction function, we used AXIN2-miR-34a-5p and WNT1-miR-34a-5p as model systems. Previously we identified both AXIN2 and WNT1 as miR-34a-5p miBridge targets and showed sequence-specific interactions between miR-34a-5p and targets in 5′ UTR and 3′ UTR sites. Here, we used HCT116 Dicer-null cell lines to minimize any other miR effects in target gene regulation. FIG. 6A shows the schematics of AXIN2 and miR-34a-5p triBridge sites. Using full length AXIN2 UTRs, we checked UTR zipper effects on loss of function and gain of function of miR-34a-5p. FIG. 6B presents the gain of miR-34a function with the increase of the number of UTR zipper interaction site, while FIG. 6C presents the loss of miR-34a function with removing the UTR zipper interaction site, without changing miR-34a-5p and target gene interaction sequences. Since all these constructs have conventional 3′ UTR interaction sites with miR-34a-5p, these gain/loss of functions of miR-34a-5p arise solely due to the 5′ UTR sequence changes. Note that we could not achieve gain/loss of function of miR-34a-5p related to this site under the same experimental conditions (the amount of transfected miR-34a differed between FIG. 6B and FIG. 6C) probably due to the limited miR-34a-5p functional range for this site.

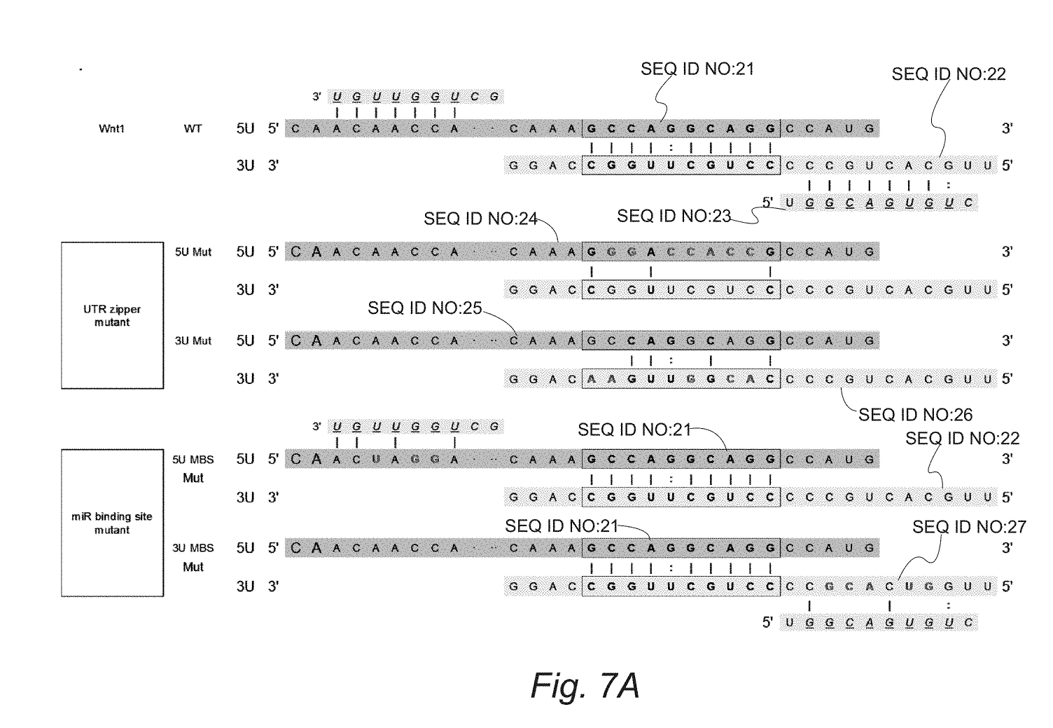

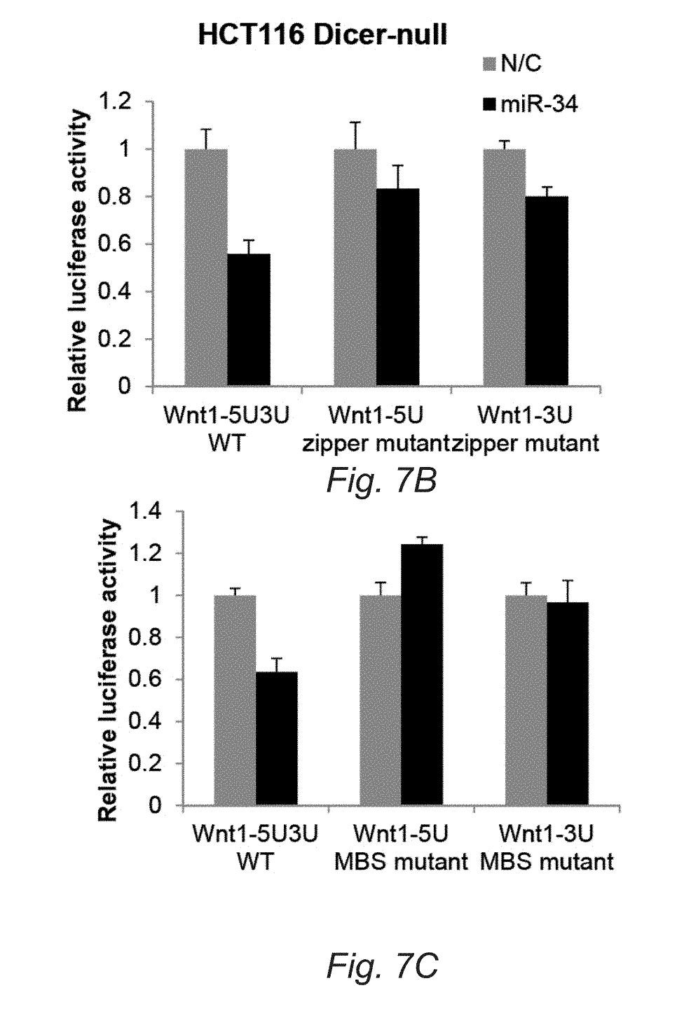

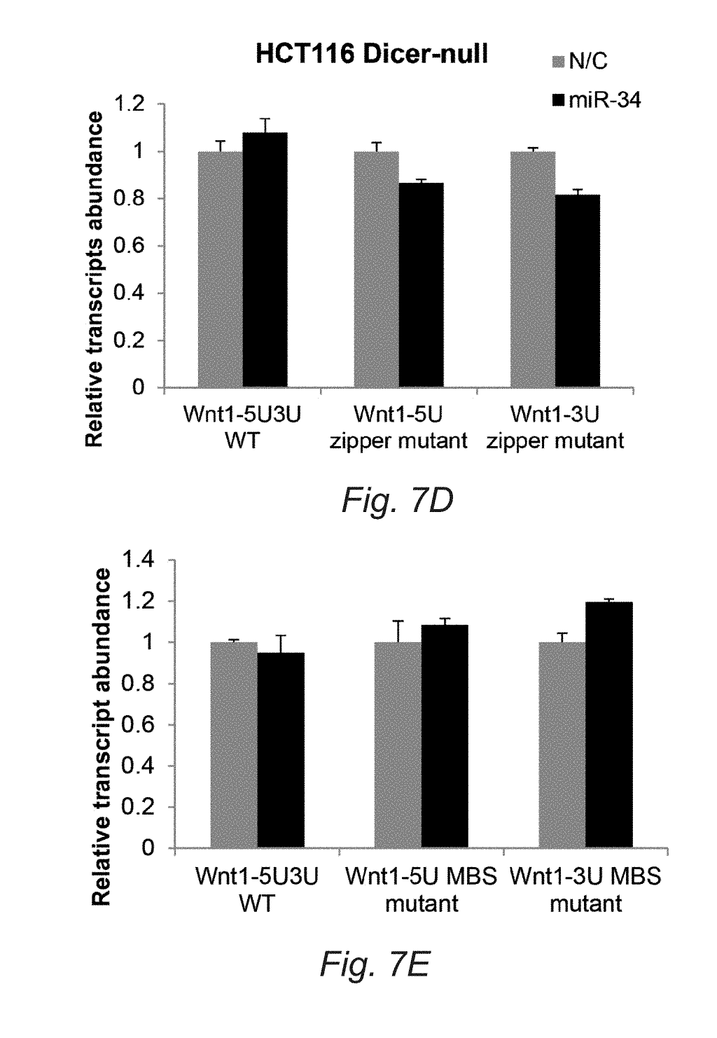

To eliminate other unknown translation regulation in 5′ UTR and to further detail the UTR zipper effects, we prepared WNT1 UTR-mimicking constructions which change the sequences of 5′ UTR or 3′ UTR at 5U=3U interaction sites and UTR=miR interaction sites. FIG. 7A provides a schematic of UTR zipper mutant and miR bonding site. FIGS. 7B and 7C show luciferase activity and relative transcript abundance using wt and mut sequences in UTRs. When both 5U and 3U zipper sites were intact, luciferase protein abundance was reduced by the miR-34a-5p, but not its transcript abundance. When either of 5U and 3U zipper site was mutated, translation repression by the miR-34a-5p is reduced, while the luciferase protein reduction amount due to miR-34a-5p corresponded to the reduction degree of its transcript. Therefore, the zipper sites contribute to translation repression rather than transcript degradation in this example. This differs from the miR-34-binding site mutations, where no transcript degradation nor protein repression occurred.

Though high throughput sequencing technology moving into medical practice, catalogues of disease-associated SNPs are mostly limited to protein coding regions. Outside of these regions, only transcription activation sites and miR interaction sites have been seriously explored. Our UTR zipper sites can provide an additional search space for functionally useful SNPs. First, we searched for known WNT1 SNPs using 1000 genome dataset and found that rare SNP rs190998135 exists in our UTR zipper site. A total four out of 2504 individuals have rs190998135, all these individuals (two males and two females) from the CLM (Colombians from Medellin, Columbia) population, from unrelated families.

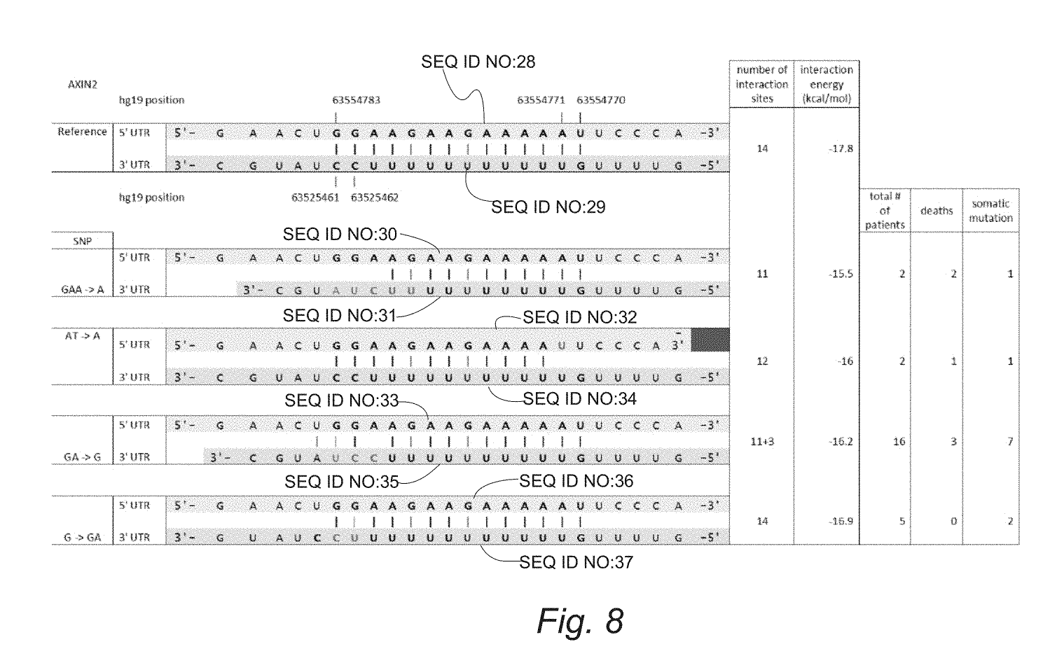

Since the UTR zipper sites of AXIN2 and WNT1 are functional, we wondered whether any SNPs of UTR zippers in these genes might be associated with diseases. We downloaded somatic mutation data of colon adenocarcinoma (COAD) and lung adenocarcinoma (LUAD) from The Cancer Genome Atlas (TCGA) and analyzed UTR mutations of AXIN2 and WNT1. Baylor College of Medicine (BCM) center has COAD SNP data from paired tumor and normal tissues of 217 patients. Among them, 40 patients have a total of 47 AXIN2 or WNT1 UTR SNPs without reference SNP numbers. Among 47 UTR SNPs, 25 of them correspond to our AXIN2 UTR zipper sites, the genome position of such SNPs in 23 patients being chr17:63525462 (3′ UTR zipper site) and that in 2 patients being chr17:63554770 (matching the 5′ UTR zipper site of the 23 patients' SNPs in the 3′ UTR as shown in FIG. 8). 11 of these 25 SNPs are somatic mutations, while only 5 of the other 22 SNPs of AXIN2 or WNT1 are somatic mutations. In terms of the survival rate of the 25 patients having these 25 SNPs, six died. Compared to 28 deaths out of the total 215 patients for which vital records were available, the death rate among these 25 patients is higher (hypergeometric distribution p-value=0.057). Though the SNP position is the same, different SNPs exist, as shown in FIG. 8. The resulting UTR zipper interactions vary depending on the type of SNPs. Interestingly, the degree of UTR zipper disruption seems to have a positive relation with the death rate: G to GA changes rarely disrupt the zipper interaction, with no death among the five individuals, while GAA to A disrupts the most, with both individuals dying. Due to the small sample size in each subgroup, follow-up study with additional samples are needed. Since hyperactive AXIN2 functions are often found in colon cancer, it is quite astonishing that more than 10% of COAD patients have AXIN2 UTR zipper SNPs. Such homogeneous and abundant AXIN2 UTR SNPs were not seen in the LUAD patient samples. There are total about 2% range of LUAD patients have AXIN2 UTR SNPs including UTR zippers at the chr17:63525462 SNPs.

In this study, we showed with genome-wide computational analyses that direct UTR-UTR interactions of the same mRNA are abundant, especially with nearby miRNA interacting sites. Using AXIN2-miR-34a-5p and WNT1-miR-34a-5p testing pairs, we demonstrated that the UTR zipper is functional and contributes to translation repression. In addition to identifying known rare SNPs in the WNT1 3′ UTR zipper site, we could associate novel SNPs in the AXIN2 UTR zipper which exist abundantly in colon cancer somatic mutations with patient survival. This new type of RNA interaction will help clarify global RNA-RNA network and serve as a valuable tool to identify novel SNPs associated with diseases.

Methods

miBridge Target Prediction

We began by constructing a database of all potential 5′ UTR to 3′ end of miRNA and 3′ UTR to 5′ end of miRNA interaction sites, or miBridge sites. All human mature miRNAs were downloaded from miRBase (Release 19) and all 5′ and 3′ UTR human sequences of RefSeq were downloaded from UCSC genome browser. An update of our miRNA data was attempted from miRBase release 19 to release 20, but was halted due to unanticipated time requirements; however, this did result in the 5 miRNA removed in release 20 to be removed from our results. We then constructed two fasta format files from miRNA sequences; one contained all the 5′ halves of the mature miRNAs while the other contained the 3′ halves. Interaction sites between the 3′-end of miRNAs and the 5′ UTR as well as those between the 5′-end of miRNA and the 3′ UTRs were identified through RNAhybrid with a parameter of −e-13 (hybridization energy cutoff value). For the 5′-end of miR and the 3′ UTR interaction, an additional parameter of −f 2,7 (requiring interactions of miR positions from 2 to 7, seed region) was set. The results from these two RNAhybrid runs were further filtered for 7 or more consecutive nucleotide matches. miBridge targets were identified if both UTRs for a single transcript interacted with a single miRNA.

Discovering 3′ UTR to 5′ UTR Interaction Sites Proximate to miBridge Sites Through RNAhybrid

Using the miBridge target site information, we constructed queries based on the regions flanking the miBridge target sites in the 3′ UTR. The queries were composed of the 30, 20, or 10 nucleotides, depending on the miBridge site's proximity to the ends of the 3′ UTR, in either the downstream or upstream direction and analyzed separately. We searched interaction sites in the entire 5′ UTR of the corresponding transcripts for each upstream or downstream query, using RNAhybrid with a parameter of an energy cutoff value of −25. The interaction separation for these two interaction sites was defined as the second position subtracted from the first. Last, we created control queries through the shuffling of the nucleotides of each query, conserving the nucleotide ratios of the originals, to generate comparison “randomized” results. The distances between miR-interaction sites and 3′ UTR interaction sites of all randomized sequences were calculated in the same manner as for the real sequences case. All histograms and plots of distances between the two interaction sites were generated using R.

Statistical Calculations

Two types of values were tested for significance with a two-sided student t-test using R. The mean interaction separation for the true sequences and every set of randomized sequences were compared. In addition, the total number of interactions occurring with a separating distance of between 10 and 20 (3′ UTR interaction sites that are near but not overlapping the miRNA interaction sites) for the true data and those of every set of randomized sequences were compared.

Models and Experimentally Verified Pairs

Through the process of searching for the UTR to UTR interaction sites, we described four different scenarios concerning how the UTRs interact, depending on whether the UTR interaction site was downstream or upstream of the corresponding miBridge site. The first two instances were when we ran RNAhybrid with our queries composed of the regions downstream of the miBridge site within the 3′ UTR, and were distinguished by whether the matching 5′ UTR interaction site was upstream or downstream of the 5′ UTR miBridge site. The second two instances were characterized by when we ran RNAhybrid with our queries composed of the regions upstream of the miBridge site within the 3′ UTR, and were once again distinguished by whether the matching 5′ UTR site was upstream or downstream of the 5′ UTR miBridge site. We chose to characterize and illustrate an example of each of these four possibilities, and did so by selecting instances of transcripts and miRNAs that were listed as experimentally verified pairs, through Reporter assay or Western blot, on miRTarBase release 4.5. This was accomplished by creating a list of all the unique miRNA and transcript pairs from our data and then taking the overlap of this list and the one obtained from miRTarBase. The orientation of the triBridge to miBridge sites was also calculated by subtracting the triBridge position from the miBridge position in each UTR. This output was then filtered down until we had 12 unique miRNAs paired with 12 unique transcripts, each permutation of upstream/downstream UTR interaction represented three times.

WNT1, Reference SNP Rs190998135 Methods

We utilized the dbSNP database from NCBI to look for known WNT1 mutations located within the interaction sites from our WNT1 and miR-34a-5-p tri-bridge model. Of the 221 known human SNPs located within WNT1, a single SNP, rs190998135 was located within the interaction sites from the model. A second model was generated for this minor allele and highlights the C to G polymorphism located in the 3′ UTR of WNT1. We further investigated the population characteristics of this minor allele by downloading the variant data for chromosome 12 from the 1000 genomes project. These data showed a total of 4 out of 2504 individuals genotyped in the project with the reference SNP. Of these four individuals, there were 2 males and 2 females, all from the CLM population from unrelated families. The minor allele (G) frequency was 0.0008 and 0.021 for all populations and the CLM population alone, respectively.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Craig, A. W. B., Haghighat, A., Yu, A. T. K. & Sonenberg, N. Interaction of polyadenylate-binding protein with the eIF4G homologue PAIP enhances translation. Nature 392, 520-523 (1998).
2. Martineau, Y. et al. Poly(A)-binding protein-interacting protein 1 binds to eukaryotic translation initiation factor 3 to stimulate translation. Molecular and cellular biology 28, 6658-6667, doi:10.1128/mcb.00738-08 (2008).
3. Sherry, S. T. et al. dbSNP: the NCBI database of genetic variation. Nucleic Acids Res 29, doi:10.1093/nar/29.1.308 (2001).
4. Lammi, L. et al. Mutations in AXIN2 cause familial tooth agenesis and predispose to colorectal cancer. American journal of human genetics 74, 1043-1050, doi:10.1086/386293 (2004).
5. Liu, W. et al. Mutations in AXIN2 cause colorectal cancer with defective mismatch repair by activating beta-catenin/TCF signalling. Nature genetics 26, 146-147, doi:10.1038/79859 (2000).
6. Ajay, S. S., Athey, B. D. & Lee, I. Unified translation repression mechanism for microRNAs and upstream AUGs. BMC Genomics 11, 155-155, doi:10.1186/1471-2164-11-155 (2010).
7. Lee, I. et al. New class of microRNA targets containing simultaneous 5′-UTR and 3′-UTR interaction sites. Genome Research 19, 1175-1183, doi:10.1101/gr.089367.108 (2009).
8. Bruno, A. E. et al. miRdSNP: a database of disease-associated SNPs and microRNA target sites on 3′UTRs of human genes. BMC Genomics 13, 1-7, doi:10.1186/1471-2164-13-44 (2012).
9. Zhang, L. et al. Functional SNP in the microRNA-367 binding site in the 3′UTR of the calcium channel ryanodine receptor gene 3 (RYR3) affects breast cancer risk and calcification. Proceedings of the National Academy of Sciences of the United States of America 108, 13653-13658, doi:10.1073/pnas.1103360108 (2011).
10. Poliseno, L. et al. A coding-independent function of gene and pseudogene mRNAs regulates tumour biology. Nature 465, 1033-1038, doi:10.1038/nature09144 (2010).
11. Sumazin, P. et al. An Extensive MicroRNA-Mediated Network of RNA-RNA Interactions Regulates Established Oncogenic Pathways in Glioblastoma. Cell 147, 370-381, doi:10.1016/j.cell.2011.09.041 (2011).
12. Hansen, T. B. et al. Natural RNA circles function as efficient microRNA sponges. Nature 495, 384-388, doi:10.1038/nature11993 (2013).
13. Memczak, S. et al. Circular RNAs are a large class of animal RNAs with regulatory potency. Nature 495, 333-338, doi:10.1038/nature11928 (2013).
14. Ellwanger, D. C., Büttner, F. A., Mewes, H.-W. & Stumpflen, V. The sufficient minimal set of miRNA seed types. Bioinformatics 27, 1346-1350, doi:10.1093/bioinformatics/btr149 (2011).
15. Lewis, B. P., Shih, I. H., Jones-Rhoades, M. W., Bartel, D. P. & Burge, C. B. Prediction of mammalian microRNA targets. Cell 115, 787-798 (2003).
16. Martinez, J., Patkaniowska, A., Urlaub, H., Larmann, R. & Tuschl, T. Single-Stranded Antisense siRNAs Guide Target RNA Cleavage in RNAi. Cell 110, 563-574, doi:http://dx.doi.org./10.1016/S0092-8674(02)00908-X (2002).
17. Matranga, C., Tomari, Y., Shin, C., Bartel, D. P. & Zamore, P. D. Passenger-Strand Cleavage Facilitates Assembly of siRNA into Ago2-Containing RNAi Enzyme Complexes. Cell 123, 607-620, doi:http://dx.doi.org/10.1016/j.cell.2005.08.044 (2005).
18. Valencia-Sanchez, M. A., Liu, J., Hannon, G. J. & Parker, R. Control of translation and mRNA degradation by miRNAs and siRNAs. Genes & development 20, 515-524, doi:10.1101/gad.1399806 (2006).
19. Ma, J. B. et al. Structural basis for 5′-end-specific recognition of guide RNA by the A. fulgidus Piwi protein. Nature 434, 666-670, doi:10.1038/nature03514 (2005).
20. Song, J. J. et al. The crystal structure of the Argonaute2 PAZ domain reveals an RNA binding motif in RNAi effector complexes. Nature structural biology 10, 1026-1032, doi:10.1038/nsb1016 (2003).

What is claimed is:

1. A method comprising:
a) executing by a computer a step of receiving into computer memory data identifying an mRNA nucleotide sequence representing a gene or portion thereof, the mRNA nucleotide sequence has an upstream UTR region that is upstream of translation start site, a downstream UTR region that is downstream of translation stop site, and an open reading frame;
b) executing by the computer a step of evaluating the upstream UTR region for a first nucleotide UTR subregion that can stably hybridize to a second nucleotide UTR subregion in the downstream UTR region such that if the first UTR nucleotide subregion can stably hybridizes the second UTR nucleotide subregion a first hybridization region is identified, the first hybridization region being a UTR-UTR zipper;
c) executing by the computer a step of receiving into computer memory data identifying a miRNA nucleotide sequence, the miRNA nucleotide sequence having a 5′ miRNA section and a 3′ miRNA section;
d) executing by the computer a step of evaluating the upstream UTR region for a third UTR nucleotide subregion that is capable of stably hybridizing to at least of a subregion of the 3′ miRNA section or at least a portion of the 5′ miRNA section such that if the third UTR nucleotide subregion can stably hybridizing to at least of a subregion of the 3′ miRNA section or at least a subregion of the 5′ miRNA section a second hybridization region is identified;
e) executing by the computer a step of evaluating the downstream UTR region for a fourth UTR nucleotide subregion that is capable of stably hybridizing to at least of a subregion of the 5′ miRNA section or at least a subregion of the 3′ miRNA section such that if the fourth UTR nucleotide subregion can stably hybridizing to at least of a subregion of the 3′ miRNA section or at least a subregion of the 5′ miRNA section a third hybridization region is identified; and
f) executing by the computer a step of identifying the mRNA nucleotide sequence as a candidate for forming a miRNA-mRNA triBridge complex, the miRNA-mRNA triBridge complex being a complex formed from the mRNA nucleotide sequence and the miRNA nucleotide sequence that includes the first hybridization region, the second hybridization region and the third hybridization region where the second hybridization region and the third hybridization region form a miRNA bridge.

2. The method of claim 1 wherein in the first hybridization region the first UTR nucleotide subregion in in an opposite direction to the second UTR nucleotide subregion with respect to 5′ to 3′ direction.

3. The method of claim 1 wherein in a miRNA interaction is upstream of the first hybridization region or downstream of the first hybridization region; or upstream of the second hybridization region or downstream of the second region.

4. The method of claim 1 wherein the first hybridization region, the second hybridization region, and the third hybridization region each independently include from 5 to 15 nucleotide base pairs.

5. The method of claim 1 wherein stable hybridization is determine by a degree of complementariness.

6. The method of claim 5 wherein complementary sub-regions include at most 2 mismatches.

7. The method of claim 1 wherein stable hybridization is determined by thermodynamic criteria.

8. The method of claim 1 further comprising obtaining a biological sample form a subject and evaluating the mRNA nucleotide sequence for polymorphisms in the first nucleotide UTR subregion and/or the second nucleotide UTR subregion.

9. The method of claim 1 further comprising obtaining a biological sample form a subject and evaluating the mRNA nucleotide sequence for polymorphisms in the third nucleotide UTR subregion.

10. The method of claim 1 further comprising obtaining a biological sample from a subject and evaluating the mRNA nucleotide sequence for polymorphisms in the fourth nucleotide UTR subregion.
